(19) United States
(12) Patent Application Publication
(10) Pub. No.: US 2023/0286888 A1
(43) Pub. Date: Sep. 14, 2023

(54) ALLOYS AND METHODS FOR ENHANCED IMPURITY REMOVAL IN DISTILLATION PROCESSES

(71) Applicant: Air Company Holdings, Inc., Brooklyn, NY (US)

(72) Inventors: Stafford W. Sheehan, Tiverton, RI (US); Michael Trainor, Burlington (CA)

(21) Appl. No.: 18/040,472

(22) PCT Filed: Aug. 5, 2021
(86) PCT No.: PCT/US21/44740

§ 371 Date: Feb. 3, 2023

Related U.S. Application Data
(60) Provisional application No. 63/062,039, filed on Aug. 6, 2020.

(51) Int. Cl. C07C 29/80; B01D 3/32; B01D 3/14; B01D 3/00; B01D 17/02

(52) U.S. Cl. CPC C07C 29/80; B01D 3/324; B01D 3/143; B01D 3/009; B01D 17/02

(57) ABSTRACT

Provided herein are metal alloy components which are useful for the removal of impurities from alcohol-containing mixtures. Also provided herein are distillation apparatuses comprising the described metal alloy components, and methods for using said components and apparatuses for the removal of impurities from alcohol-containing mixtures.

### CROSS-REFERENCE TO RELATED APPLICATIONS

This application claims the benefit of priority to U.S. Provisional Patent Application No. 63/062,039, filed Aug. 6, 2020. This application is incorporated herein by reference in its entirety.

### BACKGROUND

The removal of impurities from liquid feedstocks is a critical component of industrial process chemistry. A number of methods for the removal of such impurities have been historically employed, but many of these methods suffer from disadvantages such as high cost or low efficiency. Distillation has become a commonly used technique for industrial-scale purification of liquid feedstocks, particularly alcoholic beverages. Provided herein are metal alloys and methods for enhanced impurity removal from alcohol feedstocks by reactive distillation.

### SUMMARY OF THE INVENTION

In certain aspects, provided herein are components of a distillation apparatus, the components comprising a metal or metal alloy, wherein the metal or metal alloy comprises at least one Group 11 metal. In certain embodiments, the component comprises a metal alloy, wherein the metal alloy comprises at least two Group 11 metals. In certain embodiments, the at least two Group 11 metals are selected from copper, silver, and gold. In further embodiments, the at least two Group 11 metals are copper and silver. In yet further embodiments, the metal alloy component further comprises at least one additional metal or metalloid selected from tin, zinc, aluminum, nickel, or germanium.

In certain embodiments, the alloy comprises from about 2% to about 98% silver (w/w). In further embodiments, the alloy comprises from about 2% to about 98% copper (w/w). In yet further embodiments, the alloy comprises from about 0.1% to about 90% of the additional metal or metalloid (w/w). In still further embodiments, the metal alloy is a coating disposed upon the component. In certain embodiments, the coating has a thickness from about 50 nm to about 100 microns. In further embodiments, the coating has a thickness selected from about 50 nm, about 100 nm, about 1 micron, about 5 micron, or about 100 micron.

In further aspects, provided herein are distillation apparatuses comprising the components described herein. In certain embodiments, the distillation apparatus is a continuous distillation apparatus. In further embodiments, the apparatus comprises at least one distillation column. In yet further embodiments, the at least one distillation column comprises a packing component. In still further embodiments, the packing component comprises the metal or metal alloy. In certain embodiments, the metal or metal alloy is disposed upon the packing component. In further embodiments, the metal or metal alloy is a coating disposed upon the packing component. In yet further embodiments, the coating has a thickness from about 50 nm to about 100 microns. In still further embodiments, the coating has a thickness selected from about 50 nm, about 100 nm, about 1 micron, about 5 micron, or about 100 micron.

In certain embodiments, the at least one distillation column comprises at least one plate. In further embodiments, the at least one plate comprises the metal or metal alloy. In yet further embodiments, the metal or metal alloy is disposed upon the at least one plate. In still further embodiments, the metal or metal alloy is a coating disposed upon the at least one plate. In certain embodiments, the coating has a thickness from about 50 nm to about 100 microns. In further embodiments, the coating has a thickness selected from about 50 nm, about 100 nm, about 1 micron, about 5 micron, or about 100 micron.

In certain embodiments, the at least one distillation column comprises at least one tray. In further embodiments, the at least one tray comprises the metal or metal alloy. In yet further embodiments, the metal or metal alloy is disposed upon the at least one tray. In still further embodiments, the metal or metal alloy is a coating disposed upon the at least one tray. In certain embodiments, the coating has a thickness from about 50 nm to about 100 microns. In further further embodiments, the coating has a thickness selected from about 50 nm, about 100 nm, about 1 micron, about 5 micron, or about 100 micron.

In certain embodiments, the at least one distillation column comprises a first distillation column and a second distillation column. In further embodiments, the continuous distillation system further comprises a drying column. In yet further embodiments, the drying column comprises molecular sieves.

In yet further aspects, provided herein are methods for removing at least one impurity from an impure alcohol mixture by reactive distillation, the method comprising contacting the impure alcohol mixture with the components or the distillation apparatuses described herein, thereby generating a pure alcohol mixture.

In certain embodiments, the at least one impurity comprises a sulfur-containing impurity, a nitrogen-containing impurity, or a combination thereof. In further embodiments, the at least one impurity is selected from dimethyl ethanolamine, diethylamine, diethyl ethanolamine, diisopropylamine, ethyl amine, ethylenediamine, 2-ethoxy-3,4-dihydro-1,2-pyran, isopropylamine, methylethanolamine, triethylamine, hydrogen sulfide, methanethiol, ethanethiol, propanethiol, dimethyl sulfide, dimethyl disulfide, dimethyl trisulfide, dimethyl tetrasulfide, 2,4-dithiapentane, 3,4-dithiahexane, 2,4,5-trithiahexane, 3-methylthio-2,4-dithiapentane, methylthioacetate, methylthiopropionate, methylthiobutyrate, methylthioisovalerate, methylthioisobutyrate, methional, methylthioacetaldehyde, or a combination thereof. In yet further embodiments, the pure alcohol mixture is substantially free of the at least one impurity. In still further embodiments, the pure alcohol mixture comprises less than 10 ppm impurities. In certain embodiments, the pure alcohol mixture comprises less than 0.1 ppm impurities. In further embodiments, the pure alcohol mixture comprises less than 1 ppb impurities. In yet further embodiments, the impure alcohol mixture comprises ethanol. In still further embodiments, the impure alcohol mixture consists of ethanol and the at least one impurity.

In certain embodiments, the method comprises the steps of:
a) heating the impure alcohol mixture to generate an impure alcohol vapor;
b) contacting the impure alcohol vapor with the metal alloy component to form a pure alcohol vapor; and
c) condensing the pure alcohol vapor to form the pure alcohol mixture.

In still further aspects, provided herein are methods for purifying an alcohol-containing feedstock, wherein the alcohol-containing feedstock comprises an alcohol and at least one nitrogen and/or sulfur-containing impurity, the method comprising contacting the alcohol containing feedstock with the components of the present disclosure, thereby adsorbing the at least one nitrogen and/or sulfur-containing impurity onto the metal alloy component, and producing a purified alcohol-containing feedstock.

In certain embodiments, the alcohol is ethanol. In further embodiments, the at least one nitrogen and/or sulfur-containing impurity is selected from dimethyl ethanolamine, diethylamine, diethyl ethanolamine, diisopropylamine, ethyl amine, ethylenediamine, 2-ethoxy-3,4-dihydro-1,2-pyran, isopropylamine, methylethanolamine, triethylamine, hydrogen sulfide, methanethiol, ethanethiol, propanethiol, dimethyl sulfide, dimethyl disulfide, dimethyl trisulfide, dimethyl tetrasulfide, 2,4-dithiapentane, 3,4-dithiahexane, 2,4,5-trithiahexane, 3-methylthio-2,4-dithiapentane, methylthioacetate, methylthiopropionate, methylthiobutyrate, methylthioisovalerate, methylthioisobutyrate, methional, methylthioacetaldehyde, or a combination thereof. In yet further embodiments, the purified alcohol-containing feedstock is substantially free of the at least one nitrogen and/or sulfur-containing impurity. In still further embodiments, the purified alcohol-containing feedstock comprises less than 10 ppm of the at least one nitrogen and/or sulfur-containing impurity. In certain embodiments, the purified alcohol-containing feedstock comprises less than 0.1 ppm of the at least one nitrogen and/or sulfur-containing impurity. In further embodiments, the purified alcohol-containing feedstock comprises less than 1 ppb of the at least one nitrogen and/or sulfur-containing impurity.

In certain aspects, provided herein are systems for the purification of ethanol and/or methanol from aqueous liquid streams. In certain embodiments, the system provides for removal of water and heavy impurities in a first column, removal of a methanol and light impurities in a second column, and reactive distillation in the third column. In certain embodiments, molecular sieves are used to purify and remove water from the resulting ethanol. In certain embodiments, the system utilizes continuous distillation columns using random packing or structured packing.

In certain embodiments, provided herein are methods for the purification of ethanol or methanol from aqueous liquid streams. In certain embodiments, the system provides for removal of water and heavy impurities in a first column, removal of methanol and light impurities in a second column, and reactive distillation in the third column. In certain embodiments, methods enabling the use of molecular sieves are used to purify and remove water from the resulting ethanol. In certain embodiments, the methods utilize continuous distillation columns using random packing or structured packing.

In certain embodiments, provided herein are methods for the removal of impurities in the distillation of methanol. In some embodiments, the methanol is produced and introduced into the distillation system at a concentration of approximately 64% in water. In some embodiments, the methanol contains trace light and heavy impurities. In some embodiments, the trace light and heavy impurities are removed by distillation. In some embodiments the methanol is passed through a reactive distillation column to remove trace impurities, e.g. sulfur or nitrogen containing compounds in the methanol stream.

### BRIEF DESCRIPTION OF THE DRAWINGS

FIG. 1 is a schematic of an exemplary continuous distillation apparatus in which Column 1 (100) is a distillation column that takes in alcohol-containing liquid, and separates water and heavy distillates from an alcohol mixture comprising primarily of methanol and ethanol. Column 2 (101) separates methanol and other light impurities from the alcohol mixture. Column 3 (102) contains a copper-based packing that serves as a reactive distillation column to remove trace sulfur impurities in the ethanol. Column 4 (103) contains molecular sieves, through which the purified ethanol is passed to remove water. Feedstock alcohols in water are stored in totes (104) and fed into the distillation system using a pump (105).

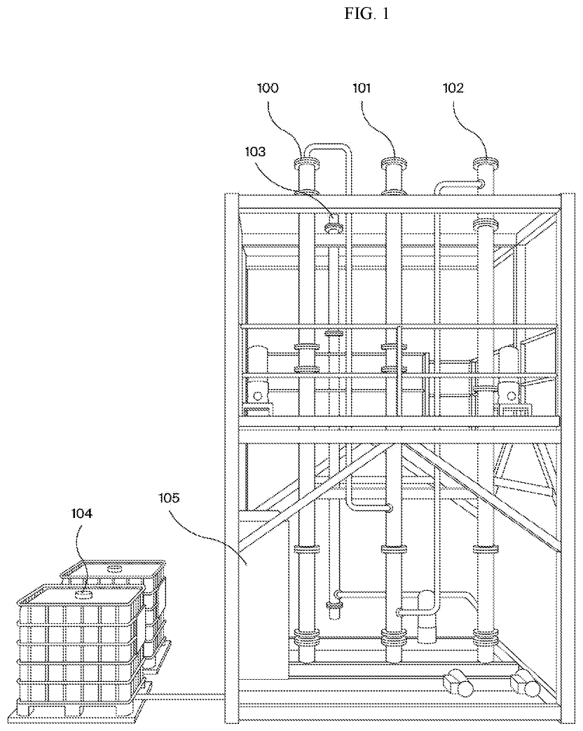

FIG. 2 is a schematic of a tray distillation column (200) comprising bubble plates (202) containing risers, slots, and caps (203) constructed with a copper/silver alloy.

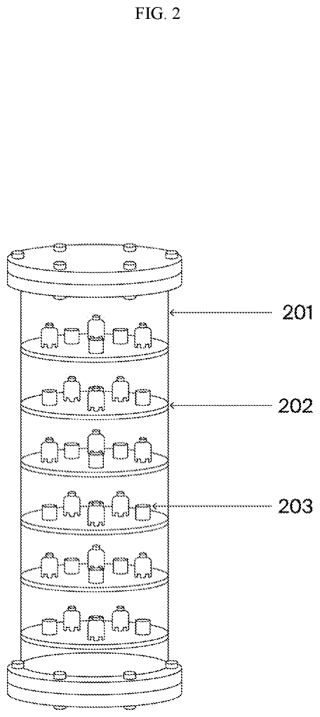

FIG. 3 is a schematic of a packed distillation column (301) comprising structured packing (302) constructed with a copper/silver alloy.

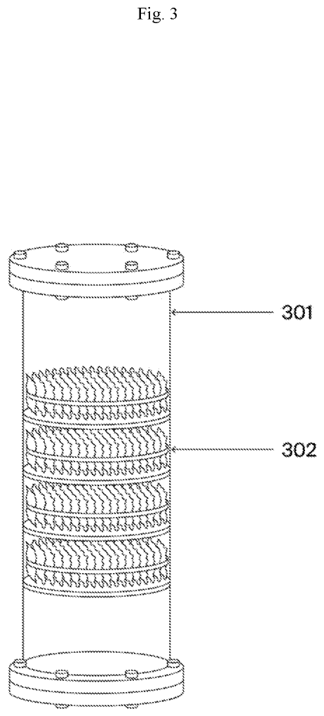

FIG. 4 is an alternate side view of the exemplary continuous distillation apparatus shown in in FIG. 1.

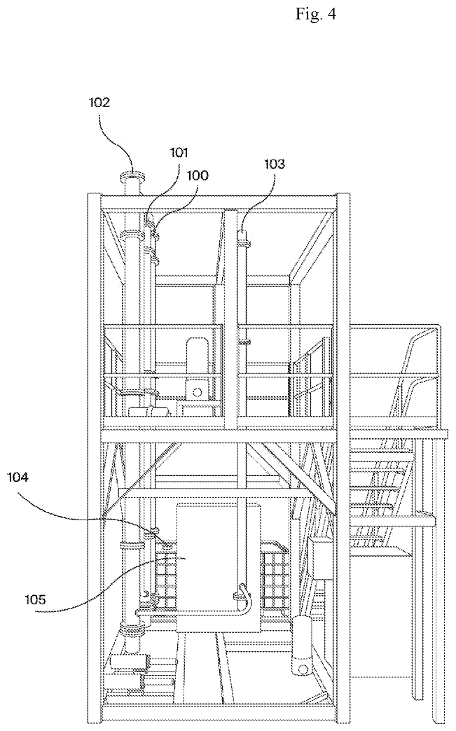

FIG. 5 is a top view of the exemplary continuous distillation apparatus shown in FIG. 1.

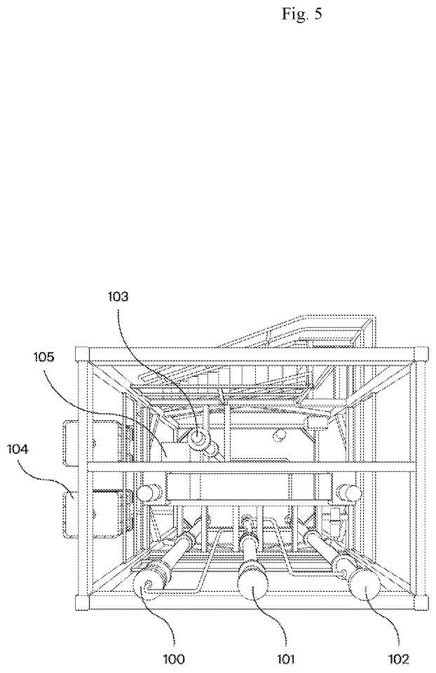

### DETAILED DESCRIPTION OF THE INVENTION

Distillation is the process by which the components of a mixture of liquids are separated by boiling and condensing selected compounds over differing spatial and/or temporal profiles. Distillation one of the oldest chemical processes in human history, with its use spanning several centuries for production of potable distilled spirits. Broadly, distillation techniques can fall into two different categories, batch (or discontinuous) distillation or continuous distillation. The first applications of distillation used batch distillation, or a batch still, which typically operates by supplying the feedstock liquid material to a kettle which is heated to the boiling point of the desired distillate or higher. The feedstock liquid material is gradually depleted as its component fractions are boiled and removed, and the resulting fractions are collected in cuts, or liquid fractions collected sequentially at different times starting with the most volatile fractions with boiling points lower than the desired distillate. The most volatile fractions are typically referred to as heads, which are followed sequentially in time by the hearts that contains the desired compound, and finally by tails which contain the least volatile fraction. A batch distillation system can be recharged to repeat the process in batches.

More recently, as distillation has become a more integral part of the chemical industry with the advent of liquid petroleum fuels that require distillation to be used in modern combustion engines, continuous distillation has become widely used. Continuous distillation typically separates the component fractions of a feedstock liquid material both spatially and temporally. In continuous distillation, the feedstock liquid material is fed continuously directly into a distillation column or columns. Typically, there are two or more liquid outlets at specific spatial intervals vertically on the distillation column, based on the heating profile of the column, that enable withdrawal of fractions at specific boiling ranges. The liquid fractions with lowest boiling points are removed at the top of the column or columns, and the fractions with the highest boiling points are removed at the bottom of the column or columns. Other hybrid forms of batch and continuous distillation have been developed, as well.

Alcoholic beverages are typically produced using batch distillation systems, both because of the history of using batch distillation systems for beverages and the lower cost of batch distillation systems combined with good control of fractional compositions. Distillation is integral to control the quality of alcoholic beverages, since many byproducts of sugar fermentation to produce ethanol, as well as several other ethanol production processes, are undesirable from either a safety perspective, or taste and quality perspective. Taste, while a highly subjective sense and product characteristic, is extremely sensitive to certain chemical compounds with tolerances far lower than even advanced chemical industrial processes. For example, the human odor threshold for methanethiol, a compound found in fermented foods such as cabbage, has been recorded as 0.06 parts per billion (ppb), on the order of tens of parts per trillion (S. Landaud, S. Helnick, P. Bonnarme, “Formation of volatile sulfur compounds and metabolism of methionine and other sulfur compounds in fermented food” Appl. Microbiol. Biotechnol. 2008, 77, 1191-1205). The presence of extremely trace and nearly undetectable quantities of several compounds, such as but not limited to methanethiol and urethane, can have a substantial adverse effect on the quality of resulting distilled spirits.

Historically, producers of distilled spirits have reduced the concentration of compounds like methanethiol and other pungent amines and thiols by producing beverage distillation systems out of copper. Copper has high heat conductivity, which helps to improve temperature control in distillation systems. Copper also readily adsorbs compounds that contain sulfur, such as sulfides and thiolates, by the spontaneous formation of copper sulfide at high rate due to the presence of heated ethanol vapors, which is a chemical reaction that occurs during spirits distillation, also known as reactive distillation. This chemical property of copper has also been studied to improve the quality of whisky by removing sulfur-containing compounds by reactive distillation, such as dimethyl trisulfide (B. Harrison, O. Fagnen, F. Jack, J. Brosnan, “The Impact of Copper in Different parts of Malt Whisky Pot Stills on New Make Spirit Composition and Aroma” J. Inst. Brew., 2011, 117, 106-112), which can also be detected by humans at concentrations in the part per trillion range. However, efficient removal of these trace impurities in ethanol is still not well implemented, as evidenced by the presence of several distilled spirits products with harsh or unpleasant tastes or odors.

Copper reacting with heated sulfur-adsorbing compounds has been investigated in the chemical industry, in hydrodesulfurization systems and reactive distillation. Hydrodesulfurization typically takes place in fixed bed reactors where gaseous products are introduced to a catalyst at high temperatures and pressures. The catalyst materials remove hydrogen sulfide from the gaseous feedstock material. The leading catalyst materials for hydrodesulfurization are cobalt molybdenum sulfide, but catalysts containing copper have also been used. These materials and processes remove sulfur to meet chemical and fuel industry standards, which have maximum concentrations on the order of 15-30 ppm. However, the sulfur removal requirements in the chemical industry require removal of much larger quantities than sulfur removal for alcoholic beverages, and do not remove trace amounts, for example, hydrodesulfurization processes decrease sulfur concentrations from 50 ppm to 2 ppm, not 1 ppm to 0.1 part per trillion as is needed for potable spirits. They also occur at highly elevated temperatures and pressures relative to spirits distillation processes, which in many cases is cost-prohibitive or unnecessary for distilled spirits.

### Metal Alloy Components

In certain aspects, provided herein are components of a distillation apparatus, the components comprising a metal or metal alloy, wherein the metal or metal alloy comprises at least one Group 11 metal. In certain embodiments, the component comprises a metal alloy, wherein the metal alloy comprises at least two Group 11 metals. In certain embodiments, the at least two Group 11 metals are selected from copper, silver, and gold. In further embodiments, the at least two Group 11 metals are copper and silver. In yet further embodiments, the metal alloy component further comprises at least one additional metal or metalloid. In still further embodiments, the additional metal or metalloid is selected from boron, silicon, germanium, arsenic, antimony, tellurium, tin, zinc, aluminum, or nickel. In yet further embodiments, the additional metal or metalloid is selected from tin, zinc, aluminum, nickel, or germanium.

In certain embodiments, the alloy comprises from about 2% to about 98% silver (w/w). In further embodiments, the alloy comprises about 5% silver (w/w). In yet further embodiments, the alloy comprises about 10% silver (w/w). In still further embodiments, the alloy comprises about 15% silver (w/w). In certain embodiments, the alloy comprises about 20% silver (w/w). In further embodiments, the alloy comprises about 25% silver (w/w). In yet further embodiments, the alloy comprises about 30% silver (w/w). In still further embodiments, the alloy comprises about 35% silver (w/w). In certain embodiments, the alloy comprises about 40% silver (w/w). In further embodiments, the alloy comprises about 45% silver (w/w). In yet further embodiments, the alloy comprises about 50% silver (w/w). In still further embodiments, the alloy comprises about 55% silver (w/w).

In certain embodiments, the alloy comprises about 60% silver (w/w). In further embodiments, the alloy comprises about 65% silver (w/w). In yet further embodiments, the alloy comprises about 70% silver (w/w). In still further embodiments, the alloy comprises about 75% silver (w/w). In certain embodiments, the alloy comprises about 80% silver (w/w). In further embodiments, the alloy comprises about 82% silver (w/w). In yet further embodiments, the alloy comprises about 84% silver (w/w). In still further embodiments, the alloy comprises about 86% silver (w/w). In certain embodiments, the alloy comprises about 88% silver (w/w). In further embodiments, the alloy comprises about 90% silver (w/w). In yet further embodiments, the alloy comprises about 92% silver (w/w). In still further embodiments, the alloy comprises about 93.5% silver (w/w). In certain embodiments, the alloy comprises about 94% silver (w/w). In further embodiments, the alloy comprises about 96% silver (w/w). In yet further embodiments, the alloy comprises about 98% silver (w/w).

In certain embodiments, the alloy comprises from about 2% to about 98% gold (w/w). In further embodiments, the alloy comprises about 5% gold (w/w). In yet further embodiments, the alloy comprises about 10% gold (w/w). In still further embodiments, the alloy comprises about 15% gold (w/w). In certain embodiments, the alloy comprises about 20% gold (w/w). In further embodiments, the alloy comprises about 25% gold (w/w). In yet further embodiments, the alloy comprises about 30% gold (w/w). In still further embodiments, the alloy comprises about 35% gold (w/w). In certain embodiments, the alloy comprises about 40% gold (w/w). In further embodiments, the alloy comprises about 45% gold (w/w). In yet further embodiments, the alloy comprises about 50% gold (w/w). In still further embodiments, the alloy comprises about 55% gold (w/w).

In certain embodiments, the alloy comprises about 60% gold (w/w). In further embodiments, the alloy comprises about 65% gold (w/w). In yet further embodiments, the alloy comprises about 70% gold (w/w). In still further embodiments, the alloy comprises about 75% gold (w/w). In certain embodiments, the alloy comprises about 80% gold (w/w). In further embodiments, the alloy comprises about 82% gold (w/w). In yet further embodiments, the alloy comprises about 84% gold (w/w). In still further embodiments, the alloy comprises about 86% gold (w/w). In certain embodiments, the alloy comprises about 88% gold (w/w). In further embodiments, the alloy comprises about 90% gold (w/w). In yet further embodiments, the alloy comprises about 92% gold (w/w). In still further embodiments, the alloy comprises about 93.5% gold (w/w). In certain embodiments, the alloy comprises about 94% gold (w/w). In further embodiments, the alloy comprises about 96% gold (w/w). In yet further embodiments, the alloy comprises about 98% gold (w/w).

In certain embodiments, the alloy comprises from about 2% to about 98% copper (w/w). In further embodiments, the alloy comprises about 2% copper (w/w). In yet further embodiments, the alloy comprises about 4% copper (w/w). In still further embodiments, the alloy comprises about 5% copper (w/w). In certain embodiments, the alloy comprises about 5.3% copper (w/w). In further embodiments, the alloy comprises about 6% copper (w/w). In yet further embodiments, the alloy comprises about 6.1% copper (w/w). In still further embodiments, the alloy comprises about 6.3% copper (w/w). In certain embodiments, the alloy comprises about 7% copper (w/w). In further embodiments, the alloy comprises about 8% copper (w/w).

In certain embodiments, the alloy comprises about 10% copper (w/w). In further embodiments, the alloy comprises about 15% copper (w/w). In yet further embodiments, the alloy comprises about 20% copper (w/w). In still further embodiments, the alloy comprises about 25% copper (w/w). In certain embodiments, the alloy comprises about 30% copper (w/w). In further embodiments, the alloy comprises about 35% copper (w/w). In yet further embodiments, the alloy comprises about 40% copper (w/w). In still further embodiments, the alloy comprises about 45% copper (w/w).

In certain embodiments, the alloy comprises about 50% copper (w/w). In further embodiments, the alloy comprises about 55% copper (w/w). In yet further embodiments, the alloy comprises about 60% copper (w/w). In still further embodiments, the alloy comprises about 65% copper (w/w). In certain embodiments, the alloy comprises about 70% copper (w/w). In further embodiments, the alloy comprises about 75% copper (w/w). In yet further embodiments, the alloy comprises about 80% copper (w/w). In still further embodiments, the alloy comprises about 85% copper (w/w). In certain embodiments, the alloy comprises about 90% copper (w/w).

In certain embodiments, the alloy comprises from about 0.1% to about 90% of the additional metal or metalloid (w/w). In further embodiments, the alloy comprises from about 0.1% to about 50% of the additional metal or metalloid (w/w). In yet further embodiments, the alloy comprises from about 0.1% to about 30% of the additional metal or metalloid (w/w). In still further embodiments, the alloy comprises from about 0.1% to about 20% of the additional metal or metalloid (w/w). In certain embodiments, the alloy comprises from about 0.1% to about 10% of the additional metal or metalloid (w/w). In further embodiments, the alloy comprises from about 0.1% to about 3% of the additional metal or metalloid (w/w). In yet further embodiments, the alloy comprises about 0.1% of the additional metal or metalloid (w/w). In still further embodiments, the alloy comprises about 0.5% of the additional metal or metalloid (w/w). In certain embodiments, the alloy comprises about 1% of the additional metal or metalloid (w/w). In further embodiments, the alloy comprises about 1.2% of the additional metal or metalloid (w/w). In yet further embodiments, the alloy comprises about 1.5% of the additional metal or metalloid (w/w). In still further embodiments, the alloy comprises about 2% of the additional metal or metalloid (w/w).

In some embodiments, the metal alloy has a copper to silver ratio of about 0.1 to 1, 0.2 to 1, 0.3 to 1, 0.4 to 1, 0.5 to 1, 0.6 to 1, 0.7 to 1, 0.8 to 1, 0.9 to 1, 1 to 1, 1 to 0.9, 1 to 0.8, 1 to 0.7, 1 to 0.6, 1 to 0.5, 1 to 0.4, 1 to 0.3, 1 to 0.2, 1 to 0.1, or lower.

### Distillation Apparatuses

In further aspects, provided herein are distillation apparatuses comprising the metal alloy components described herein. In certain embodiments, the distillation apparatus is a continuous distillation apparatus.

In certain embodiments, the apparatus comprises one or more component selected from distillation columns, drying columns, demisters, dephlegmators, valves, infusers, scrubbers, tubes, evaporators, kettles, structured packing, random packing, distillation trays, distillation plates, and other distillation system parts. In further embodiments, the apparatus comprises at least one distillation column.

In certain embodiments, the at least one distillation column comprises a packing component. In further embodiments, the packing component comprises the metal or metal alloy. In yet further embodiments, the metal or metal alloy is disposed upon the packing component. In still further embodiments, the metal or metal alloy is a coating disposed upon the packing component. In certain embodiments, the coating has a thickness from about 50 nm to about 100 microns. In further embodiments, the coating has a thickness selected from about 50 nm, about 100 nm, about 1 micron, about 5 micron, or about 100 micron.

In certain embodiments, the at least one distillation column comprises at least one plate. In further embodiments, the at least one plate comprises the metal or metal alloy. In yet further embodiments, the metal or metal alloy is disposed upon the at least one plate. In still further embodiments, the metal or metal alloy is a coating disposed upon the at least one plate. In certain embodiments, the coating has a thickness from about 50 nm to about 100 microns. In further embodiments, the coating has a thickness selected from about 50 nm, about 100 nm, about 1 micron, about 5 micron, or about 100 micron.

In still further embodiments, the at least one distillation column comprises at least one tray. In certain embodiments, the at least one tray comprises the metal or metal alloy. In further embodiments, the metal or metal alloy is disposed upon the at least one tray. In still further embodiments, the metal or metal alloy is a coating disposed upon the at least one tray. In certain embodiments, the coating has a thickness from about 50 nm to about 100 microns. In further embodiments, the coating has a thickness selected from about 50 nm, about 100 nm, about 1 micron, about 5 micron, or about 100 micron.

In certain embodiments, the at least one distillation column comprises a first distillation column and a second distillation column. In further embodiments, the continuous distillation system further comprises a drying column. In yet further embodiments, the drying column comprises molecular sieves.

In some embodiments, the present disclosure relates to components of a distillation system that contain either or both of copper and silver. In some embodiments, the distillation system is a pressurized industrial distillation system. Currently, industrial continuous distillation systems do not employ copper to react with impurities in an alcohol stream as is taught by the distilled spirits industry, which uses mostly batch distillation systems. In certain embodiments, provided herein are distillation systems containing copper alloyed or coated into other metals to act as a reactive component, while keeping the high tensile strength and structural integrity of metals typically used in industrial distillation, such as 316L stainless steel. In some embodiments, the present disclosure describes components of a distillation system that are coated with a metal or metal alloy that promotes reactive distillation for impurity removal. In some embodiments, the metal or metal alloy is coated on a steel structured packing. In some embodiments, the thickness of the copper layer is 50 nm, 100 nm, 1 micron, 5 microns, 100 microns, or higher. In some embodiments, the layer is a metal alloy containing copper. In certain embodiments, the layer or coating comprises metal atoms, such as copper, on the surface of the coating.

### Methods of Purification

In yet further aspects, provided herein are methods for removing at least one impurity from an impure alcohol mixture by reactive distillation, the method comprising contacting the impure alcohol mixture with the components or the distillation apparatuses described herein, thereby generating a pure alcohol mixture.

In certain embodiments, the at least one impurity comprises a sulfur-containing impurity, a nitrogen-containing impurity, or a combination thereof. In further embodiments, the at least one impurity is selected from dimethyl ethanolamine, diethylamine, diethyl ethanolamine, diisopropylamine, ethyl amine, ethylenediamine, 2-ethoxy-3,4-dihydro-1,2-pyran, isopropylamine, methylethanolamine, triethylamine, hydrogen sulfide, methanethiol, ethanethiol, propanethiol, dimethyl sulfide, dimethyl disulfide, dimethyl trisulfide, dimethyl tetrasulfide, 2,4-dithiapentane, 3,4-dithiahexane, 2,4,5-trithiahexane, 3-methylthio-2,4-dithiapentane, methylthioacetate, methylthiopropionate, methylthiobutyrate, methylthioisovalerate, methylthioisobutyrate, methional, methylthioacetaldehyde, or a combination thereof. In yet further embodiments, the pure alcohol mixture is substantially free of the at least one impurity. In still further embodiments, the pure alcohol mixture comprises less than 10 ppm impurities. In certain embodiments, the pure alcohol mixture comprises less than 0.1 ppm impurities. In further embodiments, the pure alcohol mixture comprises less than 1 ppb impurities. In yet further embodiments, the impure alcohol mixture comprises ethanol. In still further embodiments, the impure alcohol mixture consists of ethanol and the at least one impurity.

In certain embodiments, the method comprises the steps of:
a) heating the impure alcohol mixture to generate an impure alcohol vapor;
b) contacting the impure alcohol vapor with the metal alloy component to form a pure alcohol vapor; and
c) condensing the pure alcohol vapor to form the pure alcohol mixture.

As will be understood by one of skill in the art, at the time when the impure alcohol mixture or the impure alcohol vapor comes into contact with the metal alloy component, it may be in a purely liquid state, a purely vapor state, or in a mixture of the two physical states, depending on the location of the alloy component in the distillation apparatus.

In still further aspects, provided herein are methods for purifying an alcohol-containing feedstock, wherein the alcohol-containing feedstock comprises an alcohol and at least one nitrogen and/or sulfur-containing impurity, the method comprising contacting the alcohol containing feedstock with the component of the present disclosure, thereby adsorbing the at least one nitrogen and/or sulfur-containing impurity onto the metal alloy component, and producing a purified alcohol-containing feedstock.

In certain embodiments, the alcohol is ethanol. In further embodiments, the at least one nitrogen and/or sulfur-containing impurity is selected from dimethyl ethanolamine, diethylamine, diethyl ethanolamine, diisopropylamine, ethyl amine, ethylenediamine, 2-ethoxy-3,4-dihydro-1,2-pyran, isopropylamine, methylethanolamine, triethylamine, hydrogen sulfide, methanethiol, ethanethiol, propanethiol, dimethyl sulfide, dimethyl disulfide, dimethyl trisulfide, dimethyl tetrasulfide, 2,4-dithiapentane, 3,4-dithiahexane, 2,4,5-trithiahexane, 3-methylthio-2,4-dithiapentane, methylthioacetate, methylthiopropionate, methylthiobutyrate, methylthioisovalerate, methylthioisobutyrate, methional, methylthioacetaldehyde, or a combination thereof. In yet further embodiments, the purified alcohol-containing feedstock is substantially free of the at least one nitrogen and/or sulfur-containing impurity. In still further embodiments, the purified alcohol-containing feedstock comprises less than 10 ppm of the at least one nitrogen and/or sulfur-containing impurity. In certain embodiments, the purified alcohol-containing feedstock comprises less than 0.1 ppm of the at least one nitrogen and/or sulfur-containing impurity. In further embodiments, the purified alcohol-containing feedstock comprises less than 1 ppb of the at least one nitrogen and/or sulfur-containing impurity.

In some embodiments wherein batch (discontinuous) distillation is used, the present disclosure comprises a distillation method wherein an impure alcohol mixture is charged into a kettle. The kettle size may be 1 gallon, 5 gallons, 10 gallons, 50 gallons, 250 gallons, 500 gallons, 1000 gallons, or higher. The kettle is agitated to achieve heat uniformity by either a spinning blade attached to a motor, or by recirculation of the distillate liquid. In some embodiments, the kettle is heated by steam, in others, the kettle is heated by hot oil, in others, the kettle is directly heated electrically. In other embodiments, the kettle is heated by natural gas, oil, or coal. In some embodiments, the kettle is heated by waste heat from another process. The distillation system is supplied heat, regardless of the source.

In some embodiments, the kettle is heated to a temperature of about 50° C., 60° C., 70° C., 80° C., 90° C., 100° C., 110° C., 130° C., 150° C., or higher. The vaporous alcohol resulting from boiling the components of the mixture with boiling point less than the temperature of the kettle is passed into a vertically oriented distillation column, wherein the heated and vaporous alcohol is contacted with a plate or tray, which is comprised of a metal alloy that absorbs impurities from the vaporous alcohol. The impurities can contain either sulfur or nitrogen. In some embodiments, the metal alloy can be present at any point in the distillation system wherein vaporous alcohol is contacted with it.

In some embodiments wherein continuous distillation is used, the present disclosure relates to a distillation method wherein an alcohol-containing mixture is introduced into a heated distillation column. In some embodiments, the alcohol-containing mixture is introduced into a heated distillation column at or near its boiling point. The entry point at which the alcohol containing mixture is introduced to the column is called the feed plate, and the plates above the feed plate constitute the rectifying section while the plates below the feed plate constitute the stripping section. In some embodiments, the rectifying section is heated while the stripping section is cooled or heated to a lower temperature than the rectifying section. In some embodiments, both or either of the rectifying and stripping section is comprised of plates, structured packing, or any other packing that are comprised of a metal alloy. In some embodiments, the metal alloy can be present at any point in the distillation system wherein vaporous alcohol is contacted with it. In some embodiments, the metal alloy is structured to have maximum surface area.

In some embodiments, the distillation system is a component of a separation system that includes an initial distillation step, followed by condensation in a condenser, followed by dehydration in a molecular sieve system, followed by a finishing distillation step. In some embodiments, the distillation system is a component of a system that includes an initial distillation step and a finishing distillation step.

In some embodiments, the desired product compound distilled from the alcohol mixture is ethanol. In some embodiments, the desired product compound distilled from the alcohol mixture is methanol. In some embodiments, the desired product compound distilled from the alcohol mixture is an isomer of propanol, such as n-propanol or isopropanol. In some embodiments, the desired product compound distilled from the alcohol mixture is an isomer of butanol. In some embodiments, the desired product compound distilled from the alcohol mixture is an isomer of pentanol.

In some embodiments, the desired product compound distilled from the alcohol mixture is ethanol to be used as a component in distilled spirits, or any other alcoholic beverage. In some embodiments, the desired product compound distilled from the alcohol mixture is ethanol to be used as a component in sanitizer or other drug or pharmaceutical products.

### Systems for Distillation

In yet further aspects, provided herein are systems of distillation that incorporate the reactive elements for impurity removal. In certain embodiments, the system provides for removal of water and heavy impurities in a first column, removal of a methanol and light impurities in a second column, and reactive distillation in the third column. In certain embodiments, molecular sieves are used to purify and remove water from the resulting ethanol. In certain embodiments, the system utilizes continuous distillation columns using random packing or structured packing.

Any suitable columns for removal of water and heavy impurities may be used with the systems of the present disclosure. Exemplary columns for removal of water and heavy impurities are disclosed in U.S. Pat. No. 3,813,890, which is incorporated herein by reference in its entirety. Any suitable columns for removal of methanol and light impurities may be used with the systems of the present disclosure. Exemplary columns for the removal of methanol and light impurities are disclosed in U.S. Pat. No. 3,813,890, which is incorporated herein by reference in its entirety.

In some embodiments, the system comprises one heavy impurity removal column. In some embodiments, the system comprises two or more heavy impurity removal columns. In some embodiments, the system comprises one light impurity removal column. In some embodiments, the system comprises two or more light impurity removal columns.

In some embodiments, the heavy impurity removal column is optimized to separate out water from ethanol and methanol. In some embodiments, the heavy removal column is optimized to separate out n-propanol from ethanol and methanol. In some embodiments, the heavy removal column is optimized to separate out ethanol from methanol. In some embodiments, the heavy removal column is used to separate out isopropanol from methanol.

In some embodiments, the light impurity removal column is optimized to separate methanol from ethanol. In some embodiments, the light removal column is optimized to separate out dimethyl ether from methanol. In some embodiments, the light removal column is optimized to produce pure ethanol. In some embodiments, the light removal column is optimized to produce pure methanol.

### Definitions

As used herein, the term “about” when used before a numerical value indicates that the value may vary within a reasonable range, such as within ±10%, ±5% or ±1% of the stated value.

As used herein, the terms “weight percent” or “% w/w” are meant to refer to the quantity by weight of a compound and/or component in a composition as the quantity by weight of a constituent component of the composition as a percentage of the weight of the total composition. The weight percent can also be calculated by multiplying the mass fraction by 100. The “mass fraction” is the ratio of one substance of a mass mᵢ to the mass of the total composition m_{T} such that weight percent=(mᵢ/m_{T})*100.

All ranges disclosed herein are inclusive of the recited endpoint and independently combinable (for example, the range of “from 50 mg to 500 mg” is inclusive of the endpoints, 50 mg and 500 mg, and all the intermediate values).

As used herein, the term “alloy” refers to a partial or complete solid solution of one or more elements in a metallic matrix.

### EXAMPLES

### Example 1: Removal of Impurities from Ethanol in a Batch Fractional Distillation System Using Silver/Copper Alloy Random Packing

A glass fractional distillation apparatus is assembled, consisting of a 1000 mL round bottom flask partially submerged in a stirred hot oil bath for heating. A mesh support is placed between the 1000 mL flask and a Vigreux column packed with silver/copper alloy casting grain (93.5% silver, 5.3% copper, and 1.2% germainum). A 3-way thermometer adapter, water-cooled Liebig condenser, vacuum adapter, and 500 mL receiving flask are connected to the alloy-loaded Vigreux column. The Vigreux column is wrapped in aluminum foil to minimize heat loss. The 1000 mL heated round bottom flask is charged with 500 mL of c.a. 80% ethanol and water mixture with a minor odor and taste indicative of a trace sulfurous compound, as well as a stirring bar. The hot oil bath is heated to approximately 95° C. and monitored using a temperature probe for 6 hours, after which the product purified ethanol (c.a. 95%) without trace sulfurous odor and taste is removed from the 500 mL receiving flask.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A component of a distillation apparatus, the component comprising a metal or metal alloy, wherein the metal or metal alloy comprises at least one Group 11 metals.

2. The component of claim 1, wherein the component comprises a metal alloy comprising at least two Group 11 metals.

3. The component of claim 1 or 2, wherein the at least one Group 11 metal is selected from copper, silver, and gold.

4. The component of claim 2 wherein the at least two Group 11 metals are copper and silver.

5. The component of any one of claims 1-4, wherein the component further comprises at least one additional metal or metalloid selected from tin, zinc, aluminum, nickel, or germanium.

6. The component of claim 2, wherein the alloy comprises from about 5% to about 98% silver (w/w).

7. The component of claim 2 or claim 6, wherein the alloy comprises from about 2% to about 90% copper (w/w).

8. The component of any one of claim 2, 6, or 7, wherein the alloy comprises from about 0.1% to about 90% of the additional metal or metalloid (w/w).

9. The component of any one of claims 1-8, wherein the metal or metal alloy is a coating disposed upon the component.

10. The component of claim 9, wherein the coating has a thickness from about 50 nm to about 100 microns.

11. The component of claim 9 or 10, wherein the coating has a thickness selected from about 50 nm, about 100 nm, about 1 micron, about 5 micron, or about 100 micron.

12. A distillation apparatus comprising the component of any one of claims 1-11.

13. The distillation apparatus of claim 12, wherein the distillation apparatus is a continuous distillation apparatus.

14. The distillation apparatus of claim 12 or 13, wherein the apparatus comprises at least one distillation column.

15. The distillation apparatus of claim 14, wherein the at least one distillation column comprises a packing component.

16. The distillation apparatus of claim 15, wherein the packing component comprises the metal or metal alloy.

17. The distillation apparatus of claim 15, wherein the metal or metal alloy is disposed upon the packing component.

18. The distillation apparatus of claim 17, wherein the metal or metal alloy is a coating disposed upon the packing component.

19. The distillation apparatus of claim 18, wherein the coating has a thickness from about 50 nm to about 100 microns.

20. The distillation apparatus of claim 18 or 19, wherein the coating has a thickness selected from about 50 nm, about 100 nm, about 1 micron, about 5 micron, or about 100 micron.

21. The distillation apparatus of claim 14, wherein the at least one distillation column comprises at least one plate.

22. The distillation apparatus of claim 21, wherein the at least one plate comprises the metal or metal alloy.

23. The distillation apparatus of claim 21, wherein the metal or metal alloy is disposed upon the at least one plate.

24. The distillation apparatus of claim 23, wherein the metal or metal alloy component is a coating disposed upon the at least one plate.

25. The distillation apparatus of claim 24, wherein the coating has a thickness from about 50 nm to about 100 microns.

26. The distillation apparatus of claim 24 or 25, wherein the coating has a thickness selected from about 50 nm, about 100 nm, about 1 micron, about 5 micron, or about 100 micron.

27. The distillation apparatus of claim 15, wherein the at least one distillation column comprises at least one tray.

28. The distillation apparatus of claim 27, wherein the at least one tray comprises the metal or metal alloy.

29. The distillation apparatus of claim 27, wherein the metal or metal alloy is disposed upon the at least one tray.

30. The distillation apparatus of claim 29, wherein the metal or metal alloy component is a coating disposed upon the at least one tray.

31. The distillation apparatus of claim 30, wherein the coating has a thickness from about 50 nm to about 100 microns.

32. The distillation apparatus of claim 30 or 31, wherein the coating has a thickness selected from about 50 nm, about 100 nm, about 1 micron, about 5 micron, or about 100 micron.

33. The distillation apparatus of any one of claims 14-32, wherein the at least one distillation column comprises a first distillation column and a second distillation column.

34. The distillation apparatus of any one of claims 13-33, wherein the continuous distillation apparatus further comprises a drying column.

35. The distillation apparatus of claim 34, wherein the drying column comprises molecular sieves.

36. A distillation system removing impurities from an alcohol-containing feedstock, wherein the alcohol-containing feedstock comprises an alcohol and at least one nitrogen and/or sulfur-containing impurity, the system comprising:
a heavy impurity column for removal of water and heavy impurities;
a light impurity column for removal of methanol and light impurities; and
a reactive distillation column comprising the distillation apparatus of any one of claims 12-35.

37. The distillation system of claim 36, further comprising molecular sieves for purification and removal of water.

38. The distillation system of claim 36 or 37, wherein the columns are continuous distillation columns.

39. The distillation system of claim 38, wherein the continuous distillation columns comprise random packing or structured packing.

40. The distillation system of any one of claims 36-39, wherein the system further comprises one or more additional heavy impurity columns.

41. The distillation system of any one of claims 36-40, wherein the system further comprises one or more additional light impurity columns.

42. The distillation system of any one of claims 36-41, wherein the system further comprises one or more additional reactive distillation columns.

43. The distillation system of any one of claims 36-42 where in the heavy impurity column is optimized to separate water from ethanol and methanol.

44. The distillation system of any one of claims 36-42 where in the heavy impurity column is optimized to separate n-propanol from ethanol and methanol.

45. The distillation system of any one of claims 36-42 where in the heavy impurity column is optimized to ethanol from methanol.

46. The distillation system of any one of claims 36-42 where in the heavy impurity column is optimized to isopropanol from methanol.

47. The distillation system of any one of claims 36-46 where in the light impurity column is optimized to separate methanol from ethanol.

48. The distillation system of any one of claims 36-46 where in the light impurity column is optimized to separate dimethyl ether from methanol.

49. The distillation system of any one of claims 36-46 where in the light impurity column is optimized to produce pure ethanol.

50. The distillation system of any one of claims 36-46 where in the light impurity column is optimized to produce pure methanol.

51. A method for removing at least one impurity from an impure alcohol mixture by reactive distillation, the method comprising contacting the impure alcohol mixture with the component of any one of claims 1-11, the distillation apparatus of any one of claims 12-35, or the distillation system of any one of claims 36-50, thereby generating a pure alcohol mixture.

52. The method of claim 51, wherein the at least one impurity comprises a sulfur-containing impurity, a nitrogen-containing impurity, or a combination thereof.

53. The method of claim 51 or 52, wherein the at least one impurity is selected from dimethyl ethanolamine, diethylamine, diethyl ethanolamine, diisopropylamine, ethyl amine, ethylenediamine, 2-ethoxy-3,4-dihydro-1,2-pyran, isopropylamine, methylethanolamine, triethylamine, hydrogen sulfide, methanethiol, ethanethiol, propanethiol, dimethyl sulfide, dimethyl disulfide, dimethyl trisulfide, dimethyl tetrasulfide, 2,4-dithiapentane, 3,4-dithiahexane, 2,4,5-trithiahexane, 3-methylthio-2,4-dithiapentane, methylthioacetate, methylthiopropionate, methylthiobutyrate, methylthioisovalerate, methylthioisobutyrate, methional, methylthioacetaldehyde, or a combination thereof.

54. The method of any one of claims 51-53, wherein the pure alcohol mixture is substantially free of the at least one impurity.

55. The method of any one of claims 51-54, wherein the pure alcohol mixture comprises less than 10 ppm impurities.

56. The method of any one of claims 51-55, wherein the pure alcohol mixture comprises less than 0.1 ppm impurities.

57. The method of any one of claims 51-56, wherein the pure alcohol mixture comprises less than 1 ppb impurities.

58. The method of any one of claims 51-57, wherein the impure alcohol mixture comprises ethanol.

59. The method of any one of claims 51-58, wherein the impure alcohol mixture consists of ethanol and the at least one impurity.

60. The method of any one of claims 51-59, wherein the method comprises the steps of:
a) heating the impure alcohol mixture to generate an impure alcohol vapor;
b) contacting the impure alcohol vapor with the metal alloy component to form a pure alcohol vapor; and
c) condensing the pure alcohol vapor to form the pure alcohol mixture.

61. A method for purifying an alcohol-containing feedstock, wherein the alcohol-containing feedstock comprises an alcohol and at least one nitrogen and/or sulfur-containing impurity, the method comprising contacting the alcohol containing feedstock with the component of any one of claims 1-10, thereby adsorbing the at least one nitrogen and/or sulfur-containing impurity onto the metal alloy component, and producing a purified alcohol-containing feedstock.

62. The method of claim 61, wherein the alcohol is ethanol.

63. The method of claim 61 or 62, wherein the at least one nitrogen and/or sulfur-containing impurity is selected from dimethyl ethanolamine, diethylamine, diethyl ethanolamine, diisopropylamine, ethyl amine, ethylenediamine, 2-ethoxy-3,4-dihydro-1,2-pyran, isopropylamine, methylethanolamine, triethylamine, hydrogen sulfide, methanethiol, ethanethiol, propanethiol, dimethyl sulfide, dimethyl disulfide, dimethyl trisulfide, dimethyl tetrasulfide, 2,4-dithiapentane, 3,4-dithiahexane, 2,4,5-trithiahexane, 3-methylthio-2,4-dithiapentane, methylthioacetate, methylthiopropionate, methylthiobutyrate, methylthioisovalerate, methylthioisobutyrate, methional, methylthioacetaldehyde, or a combination thereof.

64. The method of any one of claims 61-63, wherein the purified alcohol-containing feedstock is substantially free of the at least one nitrogen and/or sulfur-containing impurity.

65. The method of any one of claims 61-64, wherein the purified alcohol-containing feedstock comprises less than 10 ppm of the at least one nitrogen and/or sulfur-containing impurity.

66. The method of any one of claims 61-65, wherein the purified alcohol-containing feedstock comprises less than 0.1 ppm of the at least one nitrogen and/or sulfur-containing impurity.

67. The method of any one of claims 61-66, wherein the purified alcohol-containing feedstock comprises less than 1 ppb of the at least one nitrogen and/or sulfur-containing impurity.
